(19) United States
(12) Patent Application Publication
(10) Pub. No.: US 2026/0345232 A1
(43) Pub. Date: Nov. 5, 2026

(54) APPARATUS FOR PULSED FIELD ABLATION APPLICATIONS WITH SUPERELASTIC MATERIALS

(71) Applicant: Alpfa Medical, Inc., Palo Alto, CA (US)

(72) Inventors: Raju VISWANATHAN, Palo Alto, CA (US); Isidro GANDIONCO, Fremont, CA (US); John MADDEN, Emerald Hills, CA (US)

(21) Appl. No.: 19/710,675

(22) Filed: Jun. 17, 2026

Related U.S. Application Data
(63) Continuation of application No. PCT/US2024/061649, filed on Dec. 23, 2024.
(60) Provisional application No. 63/613,473, filed on Dec. 21, 2023.

(51) Int. Cl. A61B 18/14; A61B 18/00

(52) U.S. Cl. CPC A61B 18/1492; A61B 2018/00267; A61B 2018/00577; A61B 2018/1467

(57) ABSTRACT

The embodiments described herein include a cylindrical braided electrode including superelastic metallic strands. In some embodiments, the cylindrical braided electrode may include a system of counter-wound strands that wind helically around a central axis. In some embodiments, an electrode comprising superelastic material may be patterned, shaped, and heat treated. In some embodiments, one or more braided and/or patterned electrodes may be mounted to a catheter and configured to be disposed in a vessel of a patient wherein the one or more braided and/or patterned electrodes may conform to a restricted vessel size. In some embodiments, pulsed field ablation can be delivered by pulsing high voltage to the braided and/or patterned electrodes after placement at an anatomical site.

### CROSS REFERENCE TO RELATED APPLICATIONS

This application is a continuation of International Application No. PCT/US2024/061649, filed Dec. 23, 2024, and entitled “APPARATUS AND METHODS FOR PULSED FIELD ABLATION APPLICATIONS WITH SUPERELASTIC MATERIALS,” which claims priority to and the benefit of U.S. Provisional Patent Application No. 63/613,473, filed Dec. 21, 2023, and entitled “APPARATUS AND METHODS FOR PULSED FIELD ABLATION APPLICATIONS WITH SUPERELASTIC MATERIALS,” the disclosures of which are incorporated by reference herein.

### TECHNICAL FIELD

This application discloses embodiments of electrode-endowed catheter devices for high-voltage pulsed field ablation applications and methods of using the same.

### BACKGROUND

Irreversible electroporation, also known as pulsed field ablation, has been previously explored in the context of surgical treatment of tumors. In surgical applications, the quest for an optimal toolset is ongoing. In the context of minimally invasive approaches, much remains to be done to develop effective minimally invasive tools that are usable while at the same time ensuring localized treatment that minimizes collateral damage.

### BRIEF DESCRIPTION OF THE DRAWINGS

FIG. 1 is a schematic illustration of a cylindrical braid of metallic strands of a superelastic material, according to an embodiment.

FIG. 2 is a schematic illustration of the cylindrical braid of FIG. 1 mounted on a catheter shaft to form an electrode, according to an embodiment.

FIG. 3 shows the mounted electrode of FIG. 2 in a deformed state after insertion into a vessel or anatomical passage.

FIG. 4 shows the mounted electrode of FIG. 2 in an involuted state.

FIG. 5 shows the involution of a single strand of the mounted electrode shown in FIG. 4.

FIG. 6 is a schematic illustration of a catheter with two basket electrodes that include braid electrodes, according to an embodiment.

FIG. 7 is a schematic illustration of a catheter with two basket electrodes that include braid electrodes, and radio-opaque markers, according to an embodiment.

FIG. 8 is a schematic illustration of a catheter with two basket electrodes that include braid electrodes, and collars, according to an embodiment.

FIG. 9 is a schematic illustration of a band electrode formed by cutting a pattern into a tube of a superelastic material, according to an embodiment.

FIG. 10 is a schematic illustration of a tool for shaping a band electrode, such as the band electrode of FIG. 9, according to an embodiment.

FIG. 11 is a schematic illustration of the band electrode of FIG. 9 shaped with the shaping tool of FIG. 10 and mounted on a catheter, according to an embodiment.

FIG. 12 is schematic illustration of a catheter with two patterned, shaped electrodes mounted on a catheter shaft having an atraumatic distal end portion, according to an embodiment.

FIG. 13 is a schematic illustration of a catheter with two patterned, shaped electrodes mounted on catheter shaft having an atraumatic distal end portion, and with a collar portion of one end of each electrode unattached to the catheter shaft, according to an embodiment.

FIG. 14 is a schematic illustration of a catheter with two patterned, shaped electrodes disposed inside a vessel of a patient, according to an embodiment.

FIG. 15 is a schematic illustration of a catheter with three patterned, shaped electrodes disposed along a distal portion of the catheter, according to an embodiment.

### DETAILED DESCRIPTION

Embodiments described herein relate to systems and devices for delivering high-voltage pulsed field ablation and methods of using the same.

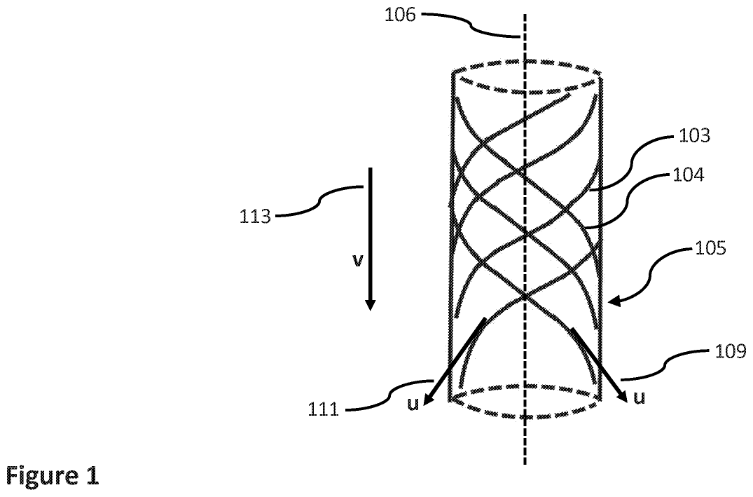

FIG. 1 schematically illustrates a cylindrical braid construction 105, according to an embodiment. In some embodiments, the cylindrical braid 105 (hereinafter, “the braid 105”) may comprise metallic strands of a superelastic material such as Nitinol. The braid 105 may include a system of counter-wound strands 103, 104 that wind helically around a central axis 106. In some embodiments, the braid 105 can include a multiplicity of such strands 103, 104. For example, the braid 105 may include 24 strands that each wind clockwise or counter-clockwise around the central axis 106 and that generally pass over and under each other to form the cylindrical braid structure 105 that holds itself together due to the braiding. In some embodiments, the number of strands in each winding direction, clockwise or counter-clockwise, (e.g., half the total number of strands in the braid 105) can be in a range between approximately 6 and 55, inclusive of all ranges and subranges therebetween.

Unit vector v, denoted by 113 in FIG. 1, defines the central axis of the helix. Examples of unit tangent vectors u along the strands 103, 104 are marked as 109 and 111 in FIG. 1. For a braid 105 with uniform winding, the winding is characterized by a pitch angle α defined by:

$$α = \frac{π}{2} - {cos}^{- 1} ( u \cdot v )$$

The pitch of the helix is the length in the longitudinal or axial direction traversed by the winding strand 103, 104 as the strand 103, 104 winds once around the central axis. Thus, a helix with a small pitch angle has a small pitch.

Such braids (e.g., braids in a cylindrical structure and/or braids having uniform winding), can have a range of stiffnesses from rigid to flexible and compressible, depending on the thickness of individual strands 103, 104, the number of strands 103, 104, and the pitch angle. While the braid 105 can deform under the action of applied forces, when made from a superelastic material such as Nitinol, the braid 105 may return to its original form (for example, a cylinder) as soon as the applied forces are released.

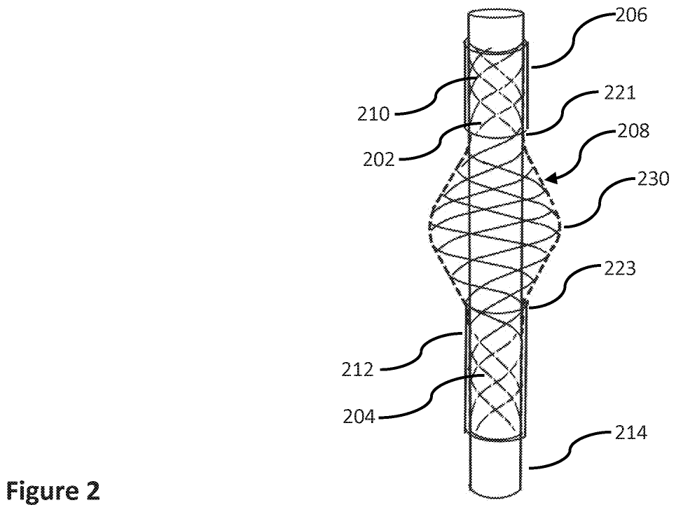

A cylindrical braid 105 can be mounted on a catheter shaft to form an electrode (e.g., a compressible electrode). As shown in FIG. 2, a braid 208 can have a shortened length when end portions 204, 210 of the braid 208 are compressed over a catheter shaft 214. The end portions 204, 210 can be held in place on the catheter shaft 214 by polymer sleeves 212, 206, respectively that can for example be heat bonded to the polymer of the catheter shaft 214. A variety of sleeve and shaft materials can be used as known to those skilled in the art, such as, for example, Nylon, Pebax, polyethylene, polyurethane, etc. Alternatively or additionally, adhesive or glue can be used to form a bond between the polymer sleeves 206, 212 and the catheter shaft 214. As shown, the middle portion 230 of the braid 208 bulges out since the end portions 204, 210 have been compressed both radially and axially, the winding (e.g., of the braid strands) may emerge out from under the polymer sleeves 206, 212 at edges 221 and 223. While the winding or pitch angle in sections of braid such as 202 within the polymer sleeve 206 can be larger, as the winding progresses to the middle portion 230 of the braid 208, the pitch decreases since the braid 208 is axially compressed, before increasing again as the winding progresses to the opposite end 204 of the braid 208 (e.g., to polymer sleeve 212). In some embodiments, an electrical lead wire can be connected to the braid 208. For example, the electrical lead wire may be coupled to the braid 208 in one or both of the polymer sleeve portions 206, 212. The electrical lead wire may be coupled to the braid 208 with mechanical contact. In some embodiments, the lead wire can be soldered or welded to the braid 208 (for example with laser welding). In some embodiments, the braid electrode 208 can have a length measured along the catheter shaft 214 in the range between approximately 2 mm and approximately 55 mm, inclusive of all ranges and subranges therebetween.

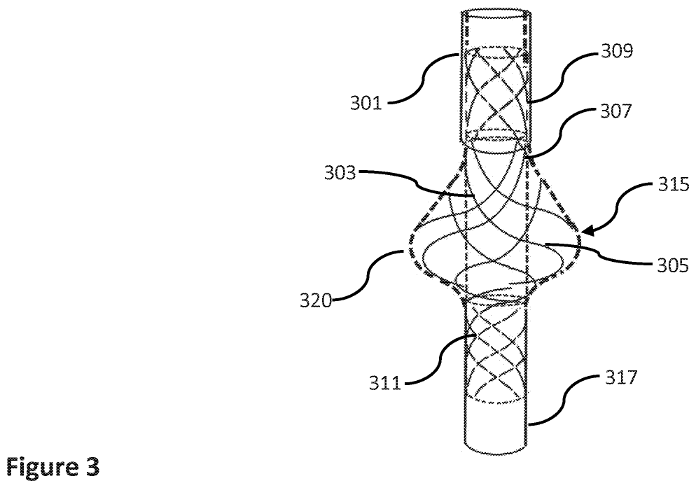

When a device with such a mounted braid 208, as described with respect to FIG. 2, is inserted into a vessel or anatomical passage (e.g., an blood vessel, lung passageway, urethra, etc.) having an inner diameter or dimension smaller than a maximum diameter or dimension of the bulge in the braid 230, the windings (e.g., the strands) can deform to conform to the vessel. When the vessel is significantly (e.g., less than 25%) smaller than the maximum diameter of the braid and not much larger than a diameter of the catheter shaft, the braid can deform significantly, as shown in FIG. 3. As shown, a catheter with catheter shaft 317 has electrode 315 (e.g., braided electrode) mounted thereon with a proximal braid portion 311 and a distal braid portion 309 attached to the catheter shaft 317. For clarity, no sleeve is shown over the braid portions 309, 311 attached to the catheter shaft 317. As the device with electrode 315 is inserted into vessel 301, the braid of the electrode 315 may deform. A portion of the braid 307 close to an entrance to or close to a diameter of vessel 301 is narrow, while more proximal portions of the braid 305 develop a compression or bulge. The winding angle of the helix in a portion 303 distal to the equator 320 (with maximum diameter of the braid) is relatively large, and as the winding progresses towards the equator 320, the winding angle decreases and portions of the braid closer to the equator such as 305 have a smaller winding angle due to the deformation. When the vessel diameter is about 25% smaller than the braid electrode diameter (maximum diameter of the bulge 230 in FIG. 2), it may not be possible for the braid 315 to move inside the vessel or passage as the catheter shaft 317 is pushed into the vessel due to the significant bulb-like deformation illustrated in FIG. 3, unless braid parameters are chosen appropriately.

Cylindrical braids generally used in medical device applications may have a pitch angle of at least 60 or 65 degrees. However, pitch angles of at least 60 to 65 degrees may prevent placement in small vessels while retaining elasticity of the electrode shape. Therefore, in embodiments described herein, the pitch angle of the initial cylindrical braid 315 (from which the braid electrode is constructed) may be less than about 55 degrees (inclusive of all values and sub-ranges), and the diameter of each strand of the braid 315 may be smaller than about 70 microns (inclusive of all values and sub-ranges) such that the braid 315 can continue to deform in a manner that enables placement in a small vessel or passage while retaining the elasticity of the overall electrode shape.

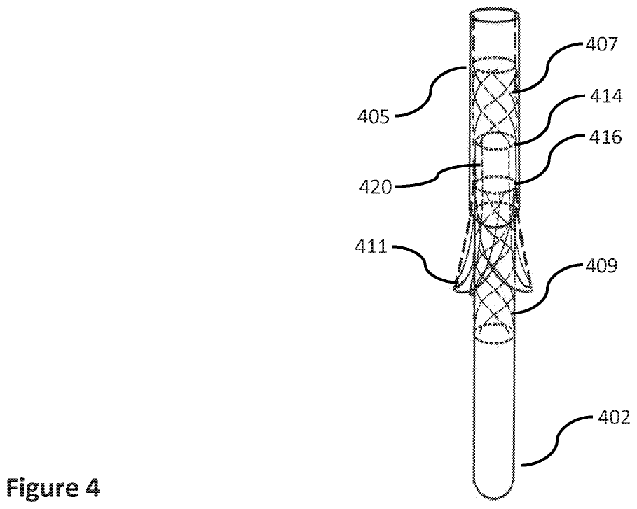

FIG. 4 illustrates a catheter with a catheter shaft 402 with a braid electrode having a proximal portion 409 and a distal portion 407 attached to the catheter shaft 402 so that an exposed/intermediate portion of the braid electrode extends between planes 416 and 414. In embodiments, the distance between planes 414 and 416, as shown in FIG. 4, can be similar to the distance between edges 221 and 223, as shown in FIG. 2. The catheter may be inserted in a vessel or passage 405 with diameter significantly (more than about 25%) smaller than the braid electrode diameter. After reaching the bulb-like configuration depicted in FIG. 3, as the catheter is pushed in, the strands 420 of the braid emerging from distal shaft-contact plane 414 involute or fold into folded portions 411 that extend on to proximal shaft-contact plane 416.

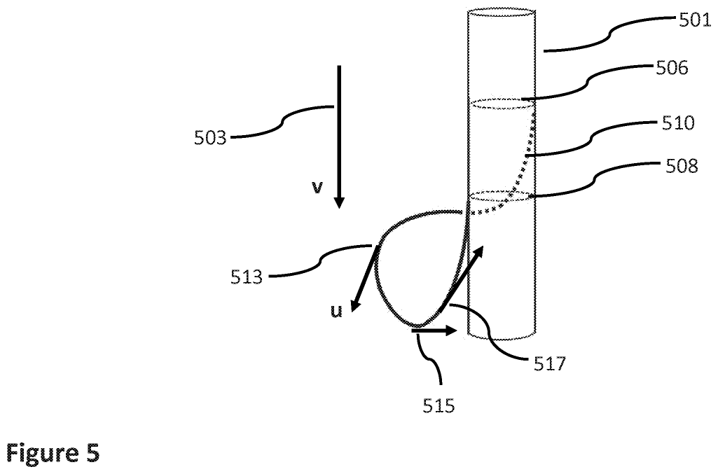

The involution of a single strand is more clearly illustrated schematically in FIG. 5. Catheter shaft 501 is shown with a single strand 510 of the braid extending from distal shaft-contact plane 506 (e.g., where a first polymer sleeve may cover the braid and hold it to the shaft, or where the braid is otherwise affixed or held to the shaft) to proximal shaft-contact plane 508 (e.g., where a second polymer sleeve may cover the braid and hold it to the shaft, or where the braid is otherwise affixed or held to the shaft). The unit vector 503 (denoted by v) defines the winding axis or long axis. As the strand 510 winds around, the unit tangent vector u along the winding at locations 513, 515 and 517 has a positive, zero and negative dot product respectively with the axis vector v. The point 515 where the tangent vector is normal to the axis vector (has zero dot product) is the point of involution of the strand 510, where the strand 510 begins to fold back on itself. When every strand or a significant majority of the strands of the braid involutes in this fashion, the braid involutes or folds on itself, enabling the catheter to easily move into the vessel or passage, e.g., even when the vessel or passage diameter is significantly smaller (more than about 25% smaller) than the braid electrode diameter.

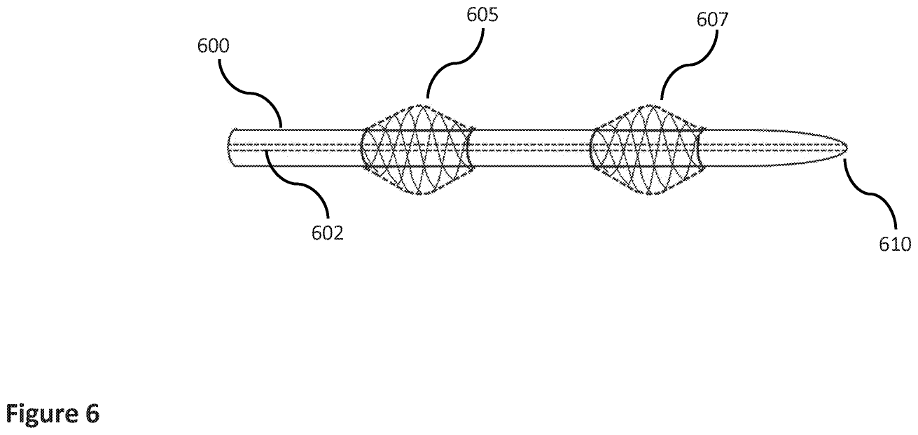

FIG. 6 schematically depicts a catheter 600 with two basket electrodes in the form of braid electrodes 605, 607, according to an embodiment. In some embodiments, each electrode 605, 607 can be connected to an insulated lead wire with the insulation capable of withstanding a voltage of at least about 700 Volts across its thickness without dielectric breakdown, inclusive of all values and sub-ranges therebetween. In some embodiments, the catheter 600 can be delivered to an anatomical site over a guidewire, and a guidewire lumen 602 runs along the length of the catheter shaft 600. Once the electrodes 605, 607 are placed/positioned at a desired anatomical site, for example a location in vasculature, pulsed field ablation can be delivered by pulsing high voltage with opposite electrical polarities to the electrodes 605, 607. In some embodiments, the catheter 600 can have an atraumatic or blunt distal tip 610 without sharp edges or corners.

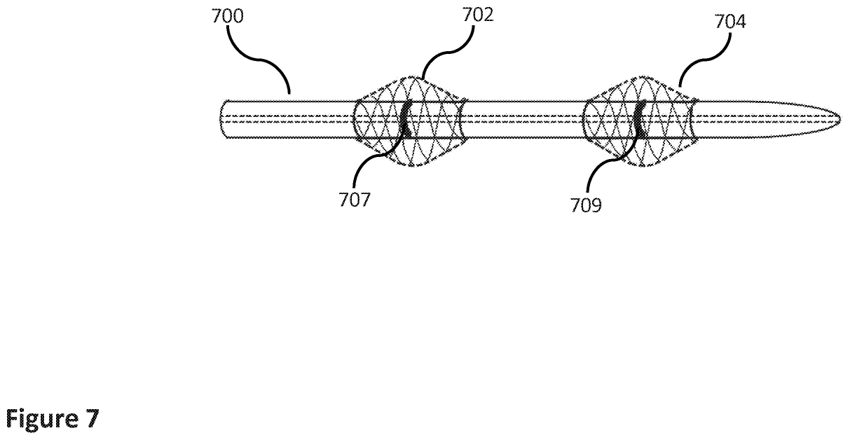

FIG. 7 schematically depicts a catheter 700 with two basket electrodes in the form of braid electrodes 702, 704, according to an embodiment. In some embodiments, one or more radio-opaque markers 707, 709 may mark the location of each electrode 702, 704 and appear dark on a fluoroscopic image. For example, the radio-opaque marker 707, 709 may include a marker band comprising radio-opaque material such as platinum, gold, tungsten or other appropriate materials and can be mounted on the shaft of the catheter 700 at or near a center point of the braid electrode 702, 704. Additionally or alternatively, the radio-opaque markers 707, 709 may mark each electrode 702, 704 at one or both ends of each electrode 702, 704 (not shown).

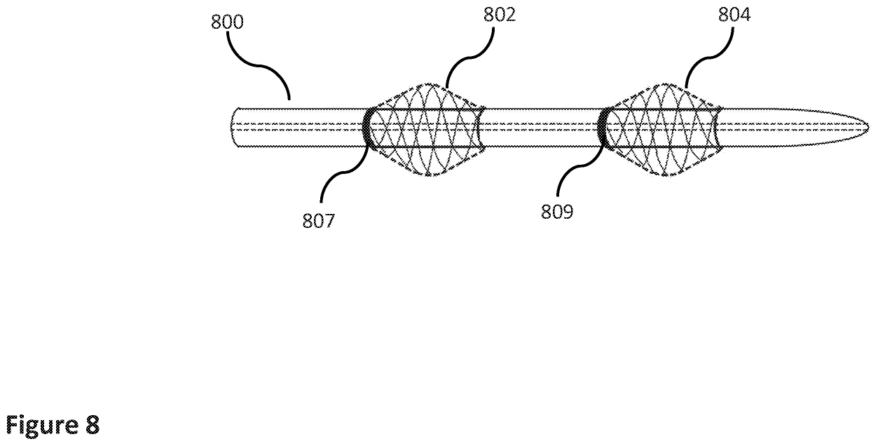

FIG. 8 schematically depicts a catheter 800 with two basket electrodes in the form of braid electrodes 802, 804, according to an embodiment. In embodiments, collars 807, 809 are attached at one end of braid electrodes 802 and 804 respectively to provide further support or integrity and/or for attachment of an electrical lead wire. In embodiments, the collars 807, 809 can also be radio-opaque or include a radio-opaque marker. In some embodiments, the collars 807, 809 can hold one end of each braid electrode 802, 804 to the shaft in a fixed manner while the other end of each braid electrode 802, 804 can be free to move relative to the shaft. In such manner, when the electrodes 802, 804 are compressed, an axial length (e.g., length of the electrode along the longitudinal axis of the shaft) can increase while allowing a radial dimension (e.g., diameter) of the electrodes to decrease.

Each of the braided electrodes described in FIGS. 1-8 can be structurally and/or functionally similar to one another. For example, the braided electrodes can have the same or similar number of strands, be compressible in the same or a similar manner, be capable of involution or folding in the same or a similar manner, and/or the like. The catheter devices including such braided electrodes can also share similar structures and/or features. For example, each can include a distal portion that is flexible and can be inserted into a vessel or anatomical passageway.

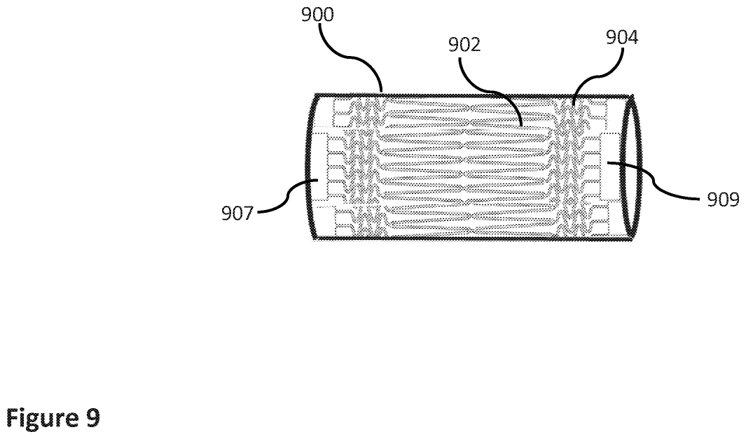

FIG. 9 is an illustration of a band electrode 900 comprising superelastic material configured as a pattern cut into a cylindrical tube. In some embodiments, the electrode 900 may have a first end and a second end defining a central portion therebetween. In some embodiments, the electrode 900 may include peripheral portions near the first end and the second end on either side of the central portion. The electrode 900 has collar portions 907, 909 at or near the first end and the second end and expandable struts 902, 904 positioned in the central portion and the peripheral portions, respectively. Such patterns (e.g., patterns including the collar portions 907, 909 and the expandable struts 902, 904) can for example be generated by a laser cutting process to cut a pre-programmed pattern into a Nitinol tube. In some embodiments, the patterned band electrode 900 can be expandable. In some embodiments, the patterned band electrode 900 can be mounted over a shaping tool and heat treated to re-form the patterned band electrode 900 into a shaped and patterned band electrode. When shaped using a shaping tool, the patterned band electrode 900 can be configured to assume that shape when unstressed (e.g., uncompressed). Additionally, the electrode 900 can be configured to be compressed (e.g., by a wall of a vessel or passageway) to assume a more tubular shape and/or to involute or fold.

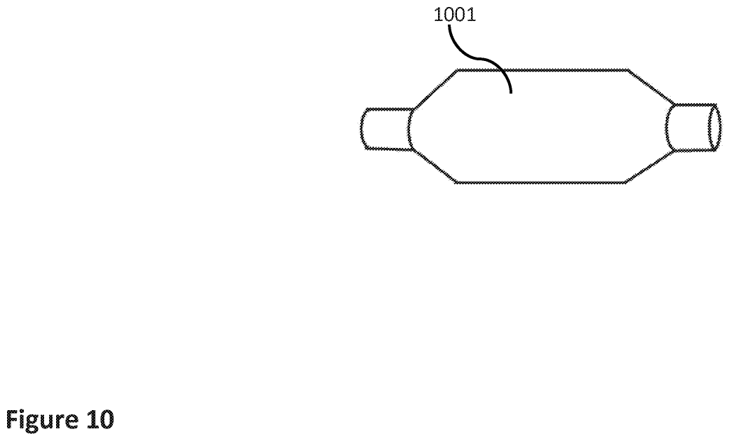

FIG. 10 depicts a trapezoidal cylinder 1001 that can serve as a shaping tool for an electrode, according to an embodiment. The electrode band 900 of FIG. 9 can be slipped over the shaping tool 1001 and heat treated in a furnace. In some embodiments, the heat treatment can comprise maintaining a pre-determined temperature for a pre-determined duration, such as for example 500 °C for 10 minutes. Upon performing the heat treatment, the electrode may take on an expanded shape as its reference shape or preferred, unstressed shape, e.g., the electrode defaults to the expanded shape in an absence of applied external forces.

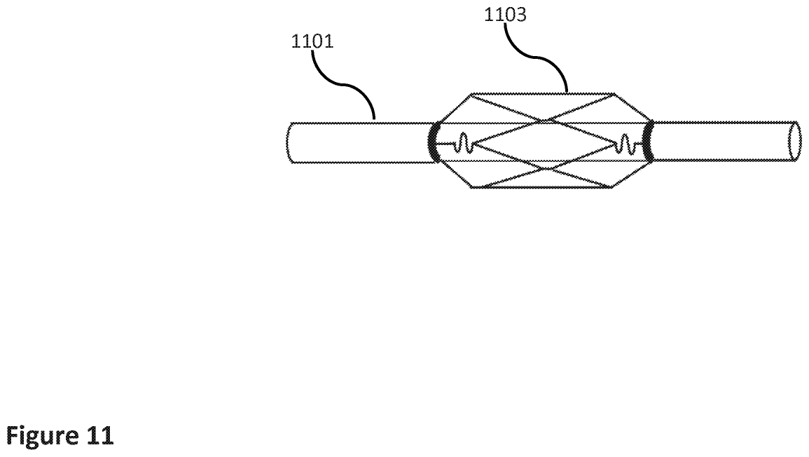

FIG. 11 shows a shaped and heat treated electrode 1103 (e.g., a compressible electrode) mounted on a catheter shaft 1101, according to an embodiment. As illustrated, the band electrode 900 of FIG. 9 has been shaped and heat treated with the shaping tool of FIG. 10 to acquire an expanded form 1103 and has been mounted on catheter shaft 1101. The electrode 1103 (e.g., after shaping and heat treatment) may retain superelastic properties. For example, the electrode 1103 can be deformed by applied forces, and when the applied forces are released, the electrode 1103 springs back or returns to the shape shown in FIG. 11. Such a patterned, shaped electrode 1103 can be passed through a tube or vessel significantly smaller (e.g., more than 25% smaller) than the electrode diameter, as the patterned shaped electrode 1103 can easily conform to the more restricted size of the tube or vessel.

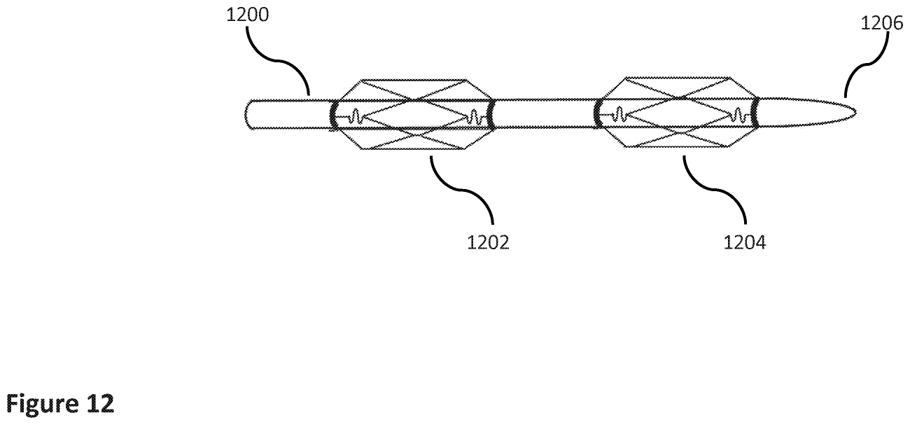

FIG. 12 illustrates a catheter 1200 with two patterned, shaped electrodes 1202 and 1204 mounted on the catheter shaft with the catheter having an atraumatic distal end portion 1206, according to an embodiment. The collar portions at both ends of each electrode 1202, 1204 can be fixed to the catheter shaft. In some embodiments, the catheter 1200 can have a guidewire lumen for delivery over a guidewire. Each electrode 1202, 1204 can be connected to an insulated lead wire with the insulation capable of withstanding a voltage of at least 700 Volts across its thickness without dielectric breakdown. Once the electrodes 1202, 1204 are placed/positioned at a desired anatomical site, for example a location in vasculature, pulsed field ablation can be delivered by pulsing high voltage with opposite electrical polarities to electrodes 1202, 1204.

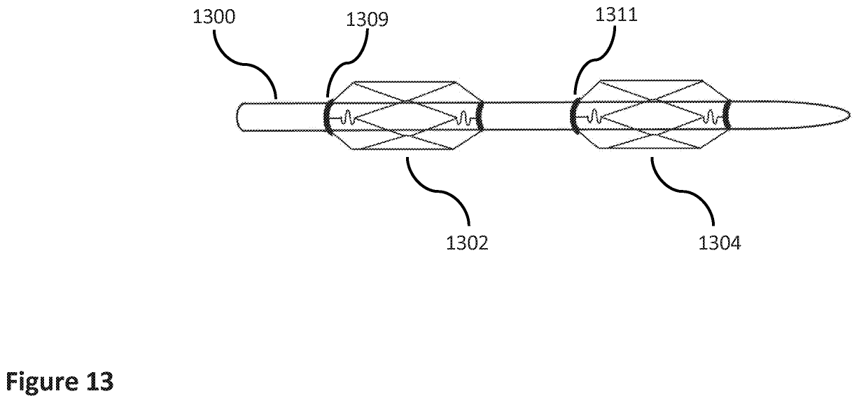

FIG. 13 illustrates a catheter 1300 with two patterned, shaped electrodes 1302, 1304 mounted on the catheter shaft with the catheter having an atraumatic distal end portion, according to an embodiment. As shown, one end or collar portion 1309, 1311 of each electrode 1302, 1304 is not fixed to the shaft. Instead, one end or collar portion of each electrode 1302, 1304 is floating or unattached (e.g., able to move relative to the shaft). With this construction, when passed through a constrained space such as a narrow vessel or anatomical passage (e.g., an blood vessel, lung passageway, urethra, etc.), the electrodes 1302, 1304 can grow in length (e.g., axial length) while reducing in diameter or radial dimension. Upon withdrawing or exiting the constrained space, the electrodes 1302, 1404 can recover to their unconstrained shape.

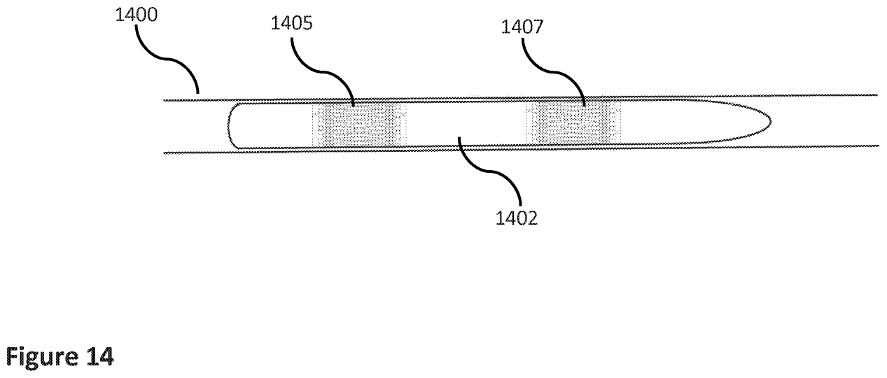

FIG. 14 illustrates a catheter 1402 with two patterned, shaped electrodes 1405, 1407 inside a vessel 1400, according to an embodiments. As shown, the electrodes 1405, 1407 compress to fit in the vessel 1400. In some embodiments, when the electrodes 1405, 1407 are compressed, the electrodes 1405, 1407 can increase in length (e.g., axial length) while reducing in diameter or radial dimension. Upon withdrawal or removal from the vessel 1400, the electrodes 1405, 1407 may regain their shape. When compressed, the electrodes can assume a shape having a diameter similar to an inside diameter of the vessel 1400.

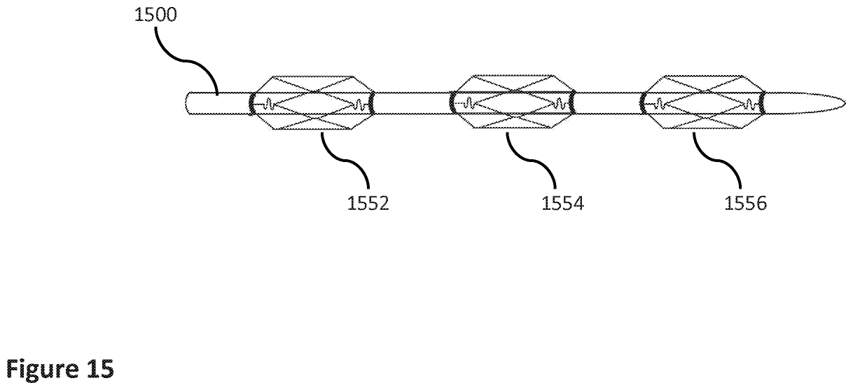

In embodiments, the catheter can have more than two electrodes. In the example shown in FIG. 15, catheter 1500 has three patterned, shaped electrodes 1552, 1554, 1556 disposed along a distal portion of the catheter. Each electrode 1552, 1554, 1556 can be connected to an insulated lead wire with the insulation capable of withstanding a voltage of at least 700 Volts across its thickness without dielectric breakdown. Once the electrodes 1152, 1554, 1556 are placed/positioned at a desired anatomical site, for example a location in vasculature, pulsed field ablation can be delivered by pulsing high voltage with opposite electrical polarities to any electrode pair or paired subsets of electrodes 1152, 1554, 1556.

Each of the patterned or shaped electrodes described in FIGS. 9-15 can be structurally and/or functionally similar to one another. For example, the electrodes can have the same or similar cut pattern, be shaped in the same or a similar manner, be capable of compressing in the same or a similar manner, and/or the like. The catheter devices including such electrodes can also share similar structures and/or features. For example, each can include a distal portion that is flexible and can be inserted into a vessel or anatomical passageway. Moreover, the braided electrodes described with respect to FIGS. 1-8 and the patterned electrodes described with respect to FIGS. 9-15 can share similar structural and/or functional features. For example, the braided electrodes and the patterned electrodes can both be examples of compressible electrodes and can be compressed in similar manners. The catheter devices including such electrodes can also share similar structures and/or features. In some embodiments, a combination of different compressible electrodes can also be used, e.g., a combination of braided and patterned electrodes.

The catheter devices of this disclosure can generally have a diameter in the range between approximately 1 mm and approximately 15 mm, inclusive of all values and subranges therebetween. The electrodes can have a maximum diameter in the range between approximately 1 mm and approximately 15 mm in their reference or unconstrained state, inclusive of all values and subranges therebetween. The electrodes can have a length in the unconstrained state in the range between approximately 2 mm and approximately 55 mm, inclusive of all values and subranges therebetween.

In embodiments, each electrical lead wire attached to an electrode can be disposed within a lumen of the catheter. In embodiments, each electrical lead wire can have a dedicated lumen. In embodiments, the braided electrodes of the present disclosure can conform to pass through a vessel with inner diameter smaller than about 75% of the diameter of the braided electrode mounted on the catheter. In embodiments, the patterned, shaped electrodes of the present disclosure can conform to pass through a vessel with inner diameter smaller than about 75% of the diameter of the patterned, shaped electrode mounted on the catheter. In embodiments, the ratio of the exposed length of an electrode of the present disclosure to its diameter is at least 1.2. While specific examples of various aspects such as electrode shapes, patterns, sizes and the like have been provided herein for exemplary purposes, it should be evident that variations and modifications of such aspects are within the scope of the present disclosure as convenient for the practice of the art.

In use, a catheter device such as those described herein can be navigated within a vessel or anatomical passageway to a target region. Once the catheter device is appropriately placed in the target region (and optionally confirmed with image guidance), electroporation ablation can be delivered in the form of a series of high voltage pulses delivered as a suitable waveform, for instance, as described in International Patent Application No. PCT/US2023/025064, filed Jun. 12, 2022, and titled “Apparatus, Systems and Methods for Soft Tissue Ablation,” the disclosure of which is incorporated herein by reference in its entirety. In some embodiments, the electrodes of the catheter device can be energized (e.g., by a generator) in a unipolar mode. In the unipolar mode, one or more of the electrodes or conductive portions on the catheter device can be configured to have one electrical polarity, while a reference patch placed on a subject has the opposite electrical polarity. In some embodiments, the electrodes of the catheter device can be energized (e.g., by a generator) in a bipolar mode. In the bipolar mode, two different electrodes or subsets of electrodes on the catheter device are energized with opposite electrical polarities. In embodiments, the voltage amplitude of the waveforms described herein can range from approximately 300 V to approximately 10,000V depending on the application, including all values and ranges therebetween.

Examples of catheter devices including electrodes formed from braids or pattern cut tubes as described herein can include, for example, those described in PCT Application No. PCT/US2023/068807, filed Jun. 21, 2023, entitled “Apparatus and Methods for Renal Denervation”, PCT Application No. PCT/US2023/075681, filed Oct. 2, 2023, entitled “Apparatus and Methods for Tissue Ablation,” U.S. Provisional Patent Application No. 63/574,637, filed Apr. 4, 2024, entitled “Catheter Apparatuses and Systems for Pulsed Electric Field Ablation Therapy,” and U.S. Provisional Patent Application No. 63/642,505, filed May 3, 2024, entitled “Balloon Catheter Apparatuses and Systems for Pulsed Electric Field Ablation Therapy,” the disclosure of each of which is hereby incorporated by reference in its entirety. For example, catheter devices described in such applications can include compressible and/or expandable electrodes, which can be formed, for example, using braiding and/or pattern cut tubes as described herein.

While diameter and/or other specific dimensional terms may be used when describing the embodiments herein, it can be appreciated that such terms are used for convenience and that other dimensional terms equivalent or similar to such terms can also change or be affected in a similar manner. For example, when describing that a diameter of an electrode may change, it can be appreciated that such a change can be characterized in terms of a change in any suitable radial dimension (e.g., a radial thickness, a width, etc.). Moreover, it can be appreciated that the drawings are not to scale, and therefore dimensions of components and/or features and distances between different components and/or features shown in the drawings may not be to scale.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” refers to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations may be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it should be understood that various changes in form and details may be made.

1. An apparatus, comprising:
a shaft including a distal portion, the distal portion being insertable into an anatomical passageway; and
a plurality of braided electrodes disposed on the distal portion,
each braided electrode of the plurality of braided electrodes having first and second ends that are coupled to the shaft,
each braided electrode of the plurality of braided electrodes being formed of a plurality of strands that wind around the shaft, the plurality of strands of each braided electrode of the plurality of braided electrodes having a central portion that is exposed and is configured to involute in response to the shaft being inserted into the anatomical passageway, the plurality of braided electrodes, when the plurality of strands are involuted, transitioning from a first diameter to a second diameter less than i) the first diameter and ii) an inner diameter of the anatomical passageway,
the plurality of braided electrodes, when the shaft is inserted into the anatomical passageway, being configured to be energized to deliver pulsed field ablation to a target region near the anatomical passageway.

2. The apparatus of claim 1, further comprising polymer sleeves configured to hold the first and second ends of each braided electrode of the plurality of braided electrodes to the shaft.

3. The apparatus of claim 1, wherein the plurality of strands of each braided electrode of the plurality of braided electrodes includes a first subset of strands that wind around the shaft in a first direction and a second subset of strands that wind around the shaft in a second direction opposite the first direction.

4. The apparatus of claim 1, wherein each strand of the plurality of strands of each braided electrode of the plurality of braided electrodes, when involuted, has an unit tangent vector along a length of the strand that starts positive, becomes zero, and then becomes negative.

5. The apparatus of claim 1, wherein, when the distal portion of the shaft is inserted into the anatomical passageway, each braided electrode of the plurality of braided electrodes is configured to transition from a first unstressed configuration to a second configuration in which the plurality of strands of that braided electrodes are involuted.

6. The apparatus of claim 5, wherein the plurality of strands of each braided electrode of the plurality of braided electrodes in the first unstressed configuration has a pitch that decreases and then increases going from a proximal end to a distal end of the braided electrode.

7. The apparatus of claim 5, wherein the distal portion of the shaft is configured to be inserted into the anatomical passageway having a diameter that is more than 25% smaller than a diameter of the plurality of braided electrodes in the first unstressed configuration.

8. The apparatus of claim 1, wherein the plurality of braided electrodes includes a first subset of one or more electrodes and a second subset of one or more electrodes, wherein the first and second subsets of electrodes are configured to be energized with opposite electrical polarities to deliver the pulsed field ablation.

9. The apparatus of claim 1, further comprising a plurality of radio-opaque markers associated with the plurality of braided electrodes, the plurality of radio-opaque markers including a radio-opaque marker disposed near a respective one of the plurality of braided electrodes.

10. The apparatus of claim 1, wherein each braided electrode of the plurality of braided electrodes includes a collar disposed at least one end of that braided electrode, the collar of each braided electrode of the plurality of braided electrodes being configured to provide structure for coupling a lead wire to that braided electrode.

11. The apparatus of claim 1, wherein the plurality of braided electrodes includes at least three braided electrodes.

12. An apparatus, comprising:
a shaft including a distal portion, the distal portion being insertable into an anatomical passageway; and
a plurality of compressible electrodes disposed on the distal portion,
the plurality of compressible electrodes, when the shaft is inserted into the anatomical passageway, being configured to transition from a first configuration in which each compressible electrode forms a preset shape having a first axial length and a first diameter that is greater than an inner diameter of the anatomical passageway to a second configuration in which each compressible electrode has a second axial length and a second diameter that is less than the inner diameter of the anatomical passageway, the second axial length being greater than the first axial length,
each compressible electrode of the plurality of compressible electrodes having a first fixed end that is coupled to the shaft and a second floating end disposed about the shaft and configured to slide along the shaft to allow each compressible electrode of the plurality of compressible electrodes to transition between the first configuration and the second configuration,
the plurality of compressible electrodes, when the shaft is inserted into the anatomical passageway, being configured to be energized to deliver pulsed field ablation to a target region near the anatomical passageway.

13. The apparatus of claim 12, wherein the plurality of compressible electrodes includes at least one patterned electrode formed from cutting a pattern into a tubular structure.

14. The apparatus of claim 13, wherein the at least one patterned electrode has been heat treated to assume the preset shape when the at least one patterned electrode is in the first configuration.

15. The apparatus of claim 12, wherein each compressible electrode of the plurality of compressible electrodes has a collar disposed at the first fixed end of that compressible electrode, the collar of each compressible electrode of the plurality of compressible electrodes configured to provide structural support for coupling a lead wire to that compressible electrode.

16. The apparatus of claim 15, wherein each compressible electrode of the plurality of compressible electrodes further includes a second collar disposed at the second floating end of that compressible electrode.

17. The apparatus of claim 12, wherein the plurality of compressible electrodes includes at least one braided electrode being formed from a plurality of strands.

18. The apparatus of claim 17, wherein the plurality of strands includes a first subset of strands that wind around the shaft in a first direction and a second subset of strands that wind around the shaft in a second direction opposite the first direction.

19. The apparatus of claim 12, wherein each compressible electrode of the plurality of compressible electrodes has a length between about 2 mm and about 55 mm.

20. The apparatus of claim 12, wherein the plurality of compressible electrodes includes a first subset of one or more electrodes and a second subset of one or more electrodes, wherein the first and second subsets of electrodes are configured to be energized with opposite electrical polarities to deliver the pulsed field ablation.

21. The apparatus of claim 23, further comprising a plurality of radio-opaque markers associated with the plurality of compressible electrodes, the plurality of radio-opaque markers including a radio-opaque marker disposed near a respective one of the plurality of compressible electrodes.

22. The apparatus of claim 12, wherein the plurality of compressible electrodes includes at least three compressible electrodes.

23. An apparatus, comprising:
a shaft including a distal portion, the distal portion being insertable into an anatomical passageway; and
a plurality of braided electrodes disposed on the distal portion,
each braided electrode of the plurality of braided electrodes having first and second ends that are coupled to the shaft,
each braided electrode of the plurality of braided electrodes being formed of a plurality of strands that wind around the shaft, the plurality of strands of each braided electrode of the plurality of braided electrodes having a pitch angle of less than about 55 degrees such that each braided electrode, when disposed in the anatomical passageway, is configured to be compressed to a diameter less than an inner diameter of the anatomical passageway,
the plurality of braided electrodes, when the shaft is inserted into the anatomical passageway, being configured to be energized to deliver pulsed field ablation to a target region near the anatomical passageway.

24. The apparatus of claim 23, wherein each braided electrode is configured to transition between a first configuration in which the braided electrode has a maximum diameter and a second configuration in which the braided electrode has the diameter less than the inner diameter of the anatomical passageway.

25. The apparatus of claim 24, wherein the first end of each braided electrode is fixed to the shaft, and the second end of each braided electrode is a floating end that is configured to slide along the shaft to allow each compressible electrode of the plurality of braided electrodes to transition between the first configuration and the second configuration.

26. The apparatus of claim 24, wherein the inner diameter of the anatomical passageway is more than about 25% smaller than a maximum diameter of the braided electrode.

27. The apparatus of claim 23, wherein a diameter of each strand from the plurality of strands is smaller than about 70 microns.

28. The apparatus of claim 23, wherein the plurality of braided electrodes includes at least one patterned electrode formed from cutting a pattern into a tubular structure.

29. The apparatus of claim 28, wherein each braided electrode from the plurality of braided electrodes is formed by laser cutting the pattern into a Nitinol tubular structure.

30. The apparatus of claim 23, wherein the plurality of braided electrodes includes a first subset of one or more electrodes and a second subset of one or more electrodes, wherein the first and second subsets of electrodes are configured to be energized with opposite electrical polarities to deliver the pulsed field ablation.
